(19) United States
(12) Patent Application Publication
(10) Pub. No.: US 2002/0190212 A1
(43) Pub. Date: Dec. 19, 2002

(54) Indirect mode imaging

(76) Inventors: David Alan Boas, Newmarket, NH (US); Andrew K. Dunn, Arlington, MA (US)

Correspondence Address: J. PETER FASSE, Fish & Richardson P.C., 225 Franklin Street, Boston, MA, 02110-2804, US

(21) Appl. No.: 10/021,862

(22) Filed: Dec. 14, 2001

Related U.S. Application Data
(60) Provisional application No. 60/256,119, filed on Dec. 15, 2000.

(51) Int. Cl. A61B 6/00

(52) U.S. Cl. 250/341.1; 600/425

(57) ABSTRACT

The invention is based on the discovery that indirect, three-dimensional images of features within turbid or dense samples can be reconstructed based on principles of radiative transport to provide higher resolution images than diffuse imaging methods, while enabling penetration depths significantly greater than microscopic imaging methods. The new indirect mode imaging methods enable one to “see” into turbid or dense samples, such as tissue in living animals or humans, ceramics, plastics, liquids, and other materials, to a depth of 50 μm to 10 mm or more, with higher resolution than diffuse imaging methods.

### CROSS-REFERENCE TO RELATED APPLICATION

This application claims priority from U.S. Provisional Patent Application Serial No. 60/256,119, filed on Dec. 15, 2000, which is incorporated herein by reference in its entirety.

### STATEMENT AS TO FEDERALLY SPONSORED RESEARCH

This invention was made with Government support under NIH 1 P41 RR 14075 and NCI T32 CA09362 awarded by the National Institutes of Health. As a result, the Government has certain rights in the invention.

### TECHNICAL FIELD

This invention relates to optical imaging, and more particularly to imaging of turbid or dense media.

### BACKGROUND

Biomedical optical imaging has been studied over the past decade at both the microscopic and diffuse levels. Microscopic methods such as confocal reflectance (see, e.g., Rajadhyaksha et al., J. Investigative Dermatol., 104:946, 1995) and multiphoton fluorescence microscopy (see, e.g., Denk et al., Science, 248:78, 1990) provide depth resolved, micron resolution images of tissue structure and function, but are limited to depths less than several hundred microns. Optical coherence tomography (OCT; see e.g., Huang et al., Science, 254:1178, 1991) can penetrate to greater depths (about 1.0 mm), but at the expense of lower spatial resolution (5-20 μm). The limitations on the penetration depths of these methods arise from the fact that each relies on the detection of single scattered light, and since tissues are highly scattering, the probability of detecting single scattered light through several hundred microns becomes prohibitively small.

Imaging with diffuse light on the other hand, typically yields spatial resolution on the order of several millimeters to a centimeter with penetration depths of several centimeters. Since the detected light has been scattered many times, an image reconstruction algorithm must be employed to reconstruct scattering and absorption perturbations. Various reconstruction strategies have been used (see, e.g., Arridge et al., Inverse Problems, 15(2):R41, 1999) and many are based on the diffusion approximation to the Boltzmann transport equation.

The spatial regime lying between that covered by the microscopic and diffuse techniques remains relatively unexplored. This is in part due to the complexity of the description of light interaction with tissue on a length scale of only a few scattering lengths. While microscopic methods yield direct reflectance or fluorescence maps of the tissue architecture, diffuse methods utilize a series of indirect measurements and subsequently reconstruct an image by solving the inverse problem. In this intermediate spatial regime, direct imaging of single-scattered light is not feasible.

### SUMMARY

The invention is based on the discovery that indirect, three-dimensional images of features within turbid or dense samples, such as tissues in living animals or humans, ceramics, plastics, liquids, and other materials, can be reconstructed based on principles of radiative transport to provide higher resolution images than diffuse imaging methods, while enabling penetration depths significantly greater than microscopic imaging methods.

In general, the invention features methods of indirect mode imaging of a sample by (a) illuminating the sample with optical radiation from a source; (b) receiving optical radiation emitted from the sample with one or more detectors; (c) digitizing the optical radiation received from the detectors to generate a digitized signal and transmitting the digitized signal to a processor; and (d) processing the digitized signal to reconstruct an image of spatially varying optical properties of features within the sample by performing a nonlinear minimization between the digitized data and a transport-based photon migration model.

In these methods, a “non-linear minimization” is an algorithm designed to minimize the difference between two quantities, as described in further detail below. A “transport-based photon migration model” is a model that describes the propagation of light (photons) through a turbid medium such as biological tissue, as described in further detail herein.

In these methods, the detectors can each be located at a position offset from the source, and one or more of the offsets of each detector-source pair can be different. The image δμₐ(r) can be reconstructed by calculating a matrix equation of the form y=Ax, wherein μₐ is the absorption coefficient, r is a position within the sample, x=δμₐ, y is the vector of detector signals for each source-detector pair, and Aᵢⱼ is an element of matrix A representing the product of the Green function for the transport equation (G) and the first-order (background) detector signal (L₀) for each measurement i at each voxel j. In these methods, the optical radiation can be visible, near-infrared, or other radiation, and there can be one or more sources. Alternatively, the optical radiation can be scanned from the source across the sample to simulate multiple sources. For example, 10, 15, 20, 30, 40, 50 or more detectors (or sources) can be used. The offset can be, for example, from 0.1 to 10 mm.

In another aspect, the invention features systems for indirect imaging of a sample that includes (a) a probe comprising a source optic fiber, one or more detector optic fibers, and a distal end, wherein a distal end of the source optic fiber and each of the detector optic fibers extends through and ends in the distal end of the probe; (b) an optical radiation source connected with a proximal end of the source optic fiber; (c) one or more photodetectors, each connected to a proximal end of one of the detector optic fibers, to detect and convert optical radiation from each detector optic fiber into a digital signal corresponding to the light emitted from the sample; and (d) a processor that processes the digital signal produced by the photodetectors to provide on an output device an image of spatially varying optical properties of features within the sample, wherein the image is reconstructed by performing a non-linear minimization between the digitized data and a transport-based photon migration model.

In these systems, the distal end of each detector optic fiber can be offset from the distal end of the source optic fiber, e.g., by about 0.1 to 10 mm. In addition, in this system, the processor is programmed to process the digital signal to provide an image δμₐ(r) that is reconstructed by calculating a matrix equation of the form y=Ax, wherein μₐ is the absorption coefficient, r is a position within the sample, x=δμₐ, y is the vector of detector signals for each source-detector pair, and Aᵢⱼ is an element of matrix A representing the product of the Green function for the transport equation (G) and the first-order (background) detector signal (L₀) for each measurement i at each voxel j.

As above, the one or more of the offsets of each detector-source pair can be different, the optical radiation can be near-infrared radiation, and the optical radiation can be scanned from the source optic fiber across the sample to simulate multiple sources. In some embodiments, 10, 15, 20, 30, 40, 50 or more detectors (or sources) can be used. The offset can be from 0.1 to 10 mm, and can vary form one to the other offset.

In another aspect, the invention features a probe for indirect mode imaging. The probe includes a source optic fiber, one or more detector optic fibers, and a distal end, wherein a distal end of the source optic fiber and each of the detector optic fibers extends through and ends in the distal end of the probe, and wherein the distal end of the source optic fiber is offset from each distal end of the detector optic fibers by 0.1 to 10 mm. The probe can further include a scanning mirror to scan optical radiation across a sample. The probe can have a variety of different length offsets for the one or more source-detector pairs.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The new imaging methods provide the ability to “see” into a sample, e.g., tissue, to a depth of 50 μm to 10 mm, which is far greater than is possible using microscopic imaging methods, which typically provide a view of only the surface of a sample. At the same time, the new methods provide a resolution of 10 μm to about 1 mm, which is significantly greater than is possible using diffuse imaging methods that provide useful penetration depths, but a resolution of only 5 to 10 mm. The new methods can be used in various diverse applications such as biomedical applications including functional imaging of the cerebral cortex through an intact skull, endoscopic imaging of small lesions too deep for conventional microscopic techniques to image and too small for diffuse methods to resolve, and ophthalmological imaging, e.g., of layers within the retina. Other applications include imaging of ceramics, semiconductors, and other turbid or dense materials, e.g., during processing.

A significant advantage of the technique described herein is that it enables for the first time optical imaging in the intermediate regime between microscopy and diffuse optical tomography. Other features and advantages of the invention will be apparent from the following detailed description, and from the claims

### BRIEF DESCRIPTION OF DRAWINGS

FIG. 1 is a graph illustrating the trade-off between penetration depth and resolution for various imaging methods including the new indirect mode imaging methods.

FIG. 2 is a schematic diagram of a general imaging geometry useful in the new imaging methods.

FIG. 3 is a graph of a perturbed signal computed with a first Born approximation (solid lines) and with a full perturbative Monte Carlo simulation (symbols) at source-detector separations of 500 μm and 2.0 m for an object located 1.0 m beneath the surface with δμₐ=0.1 mm⁻¹.

FIG. 4A is an end view of an illumination/detection probe useful in the new imaging methods.

FIG. 4B is a schematic diagram of an illumination/detection probe in a scanning system.

FIG. 5A is a flowchart illustrating the steps used to acquire and process optical signal data in the new methods.

FIG. 5B is a flowchart illustrating the steps used to process the optical signal to reconstruct an image of a feature in the sample.

FIG. 6 is an endoscopic illumination/detection probe for use in the new methods.

FIG. 7 is a computer-based simulation of the distribution of photons for a source-detector separation of 750 μm.

FIG. 8 is a graph of normalized radial intensity at z=500 μm vs. position on the x-axis for the cases where the angular dependence is considered (“transport”—solid line) and the case where it is ignored (“diffusion”—dashed line).

FIGS. 9A and 9B are a pair of reconstructed images of a 100 μm absorbing object located at a depth of 1.0 m (9A) or 2.0 m (9B) below the surface using simulated data (δμₐ=0.1 mm⁻¹).

Like reference symbols in the various drawings indicate like elements.

### DETAILED DESCRIPTION

The invention is based on the discovery that indirect, three-dimensional images of features within turbid or dense samples can be reconstructed based on principles of radiative transport to provide higher resolution images than diffuse imaging methods, while enabling penetration depths significantly greater than microscopic imaging methods. The new indirect mode imaging methods enable one to “see” into turbid or dense samples, such as tissue in living animals or humans, ceramics, plastics, liquids, and other materials, to a depth of about 50 μm to about 10 mm (e.g., 100 μm to 5 mm, or 500 μm to 1.0 m) or more, with higher resolution than diffuse imaging methods, which provide somewhat greater penetration depths, but low resolution.

### General Methodology

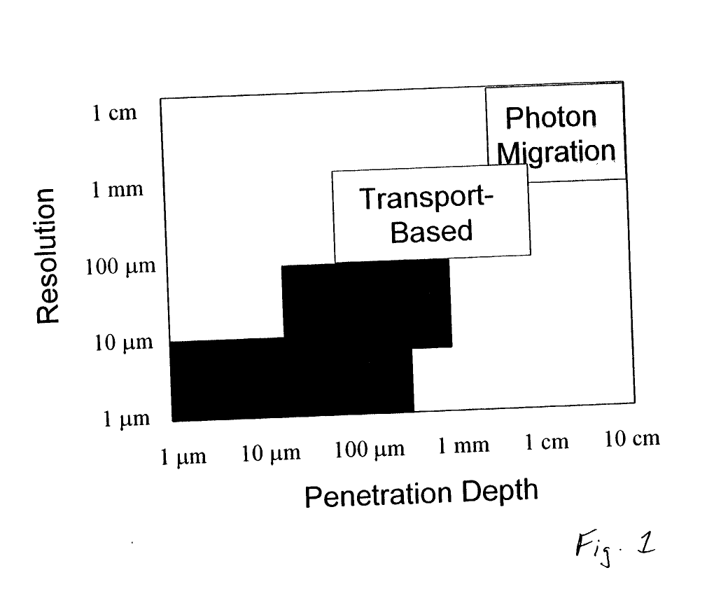

Optical imaging, and in particular biomedical imaging, has progressed over the years, but there is a significant gap in the ability to image features within a sample at a depth of between about 1 mm and 1 cm, which is a very useful range in medical and other imaging. There has always been a trade-off between penetration depth and resolution, but to date, there has been no method of imaging features at a depth of 1 mm to 1 cm with high resolution. This trade-off is summarized in the graph in FIG. 1. Although microscopy provides excellent resolution (1 to 10 μm), it cannot be used to image much below the surface of a sample (300 to 500 μm at best). On the other hand, diffuse imaging methods based on photon migration allow useful penetration depths of up to about 10 cm, but at that depth provide a resolution of only about 1 cm. Optical Coherence Tomography (OCT) has enabled better penetration depths than microscopy (up to about 0.5 to 1.0 m), but still does not provide an image of deeper features in a sample, and at the maximum penetration depth provides a resolution of about 100 μm.

The new indirect mode imaging (IMI) methods are based on the principle that images can be reconstructed based on the framework of the radiative transport equation (RTE) (see, e.g., Ishimaru et al., Wave Propagation and Scattering in Random Media, Vol. 1 (Academic Press, Inc. 1978). An important distinction between reconstruction based on the RTE and the photon diffusion equation (DE) is the treatment of the angular dependence of the scattered light within the tissue. The new methods include both indirect microscopic imaging and indirect macroscopic imaging.

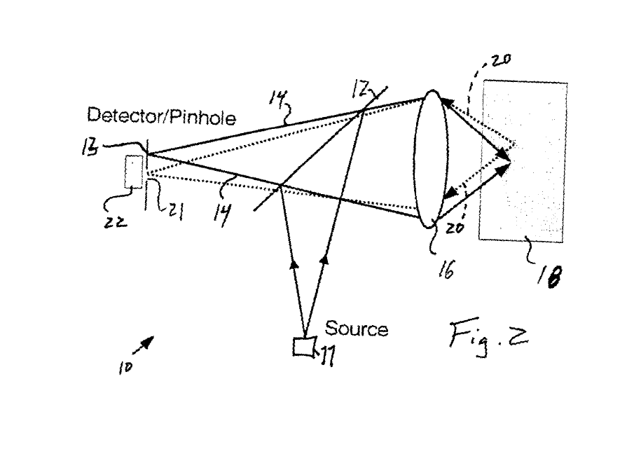

FIG. 2 illustrates a general geometry 10 used to image in the new IMI methods, in which light is multiply scattered, but not yet diffuse. A source 11 directs light to a mirror 12 and through lens 16 into target 18. Light reflected from the sample along the optical axis (beams 14) hits point 13. By translating a pinhole 21 and a detector 22, which is typically used in confocal reflectance measurements, to positions laterally displaced from the optical axis (at point 13), the pinhole 21 is imaged onto the detector 22 to a point laterally displaced from the source. Therefore, the amount of lateral displacement of the detector aperture from the source aperture determines the effective source-detector separation. This separation distance can vary from 0 to about 10 mm. Because the source and detector no longer lie in conjugate image planes, the detected light has been scattered at least one time. This scattered light is represented by dotted lines 20 in FIG. 2.

As the source-detector separation distance increases, the detected light will probe a deeper and larger region of the sample. To maximize the sensitivity of the measurement to a particular region of the sample, several parameters can be varied in the geometry of FIG. 2. For example, the source-detector separation (aperture offset, 0 to about 10 mm, e.g., 1, 2, 3, 5, or 7 mm), numerical aperture (0 to about 1.0, e.g., 0.1, 0.2, or 0.3), depth of focus into the medium (0 to about 5 mm, e.g., 1, 2, 3, or 4 mm), wavelength (typically about 400 to about 1500 nm, e.g., 570 to 650 nm or 1300 to 1500 nm), and pinhole diameter (about 100 μm to about 1.0 mm, e.g., 100, 150, 200, 250, or 300 μm, or 0.5, 0.75 or 0.9 mm can all be varied). In general, the size of the pinhole aperture will be larger than that used in confocal reflectance measurements since it will be more important to maximize the signal intensity than to provide a large degree of spatial filtering.

Direct imaging of single-scattered light using the geometry of FIG. 2 is not feasible. Therefore, to form an image, an inverse problem must be solved as in the diffuse regime. Since the diffusion approximation is no longer valid due to the close proximity of the source and detector, a description based on the transport equation (see, e.g., Ishimaru et al., Wave Propagation and Scattering in Random Media, Vol. 1 (Academic Press, Inc. 1978)) is used.

To reconstruct an image of an absorption perturbation with a source-detector separation of only a few scattering lengths, one can use a linear reconstruction algorithm based on the first Born approximation to the radiative transport equation. In the first Born approximation, the absorption coefficient (μₐ) and scattering coefficient (μₛ) are written as a sum of a homogeneous background component and a spatially varying perturbation,

μₐ(r)=μ^{o}ₐ+δμₐ(r) and μₛ(r)=μ⁰ₛ+δμₛ(r)

The radiance (i.e., the amount of light received at the detector), L, is then expressed as the sum of a background and a perturbed component, L=L₀+L₁. For an absorption perturbation the zeroth- and first-order terms, L₀ and L₁, are given by:

L₀(r_{d}, \hat{Ω}_{d})=∫∫S(r, \hat{Ω})G(r, \hat{Ω}|r_{d}, \hat{Ω}_{d})d\hat{Ω}d³r (1)

L₁(r_{d}, \hat{Ω}_{d})=−∫∫δμₐ(r)L₀(rₛ,r, \hat{Ω})G(r, \hat{Ω}|r_{d}, \hat{Ω}_{d})d\hat{Ω}d³r (2a)

where G(r, Ω|r_{d}, Ω_{d}) is the Green function solution for the transport equation for a particular detector configuration. Perturbations in the scattering coefficient and phase function can be computed using the same approach and the first-order term for a scattering perturbation is given by

L₁(r_{d}, \hat{Ω}_{d})=−∫∫δμₛ(r)[L₀(rₛ, r, \hat{Ω})−∫L₀(rₛ, r, \hat{Ω}′)p(Ω, Ω′)dΩ′]G(r, \hat{Ω}|r_{d}, \hat{Ω}_{d})d\hat{Ω}d³r (2b)

Eqs. 2a and 2b illustrate that one of the primary differences between the new indirect mode imaging methods and imaging methods based on the diffusion approximation lies in the angular dependence of the radiance. Here, we only consider reconstruction of an absorbing object using Eq. 2a, but the same procedure can be followed to reconstruct a scattering perturbation. To reconstruct scattering perturbations, one uses exactly the same procedure but substitutes Eq. 2b for Eq. 2a. To simultaneously reconstruct an image of both absorption and scattering, one uses the summation of Eq. 2a and Eq. 2b.

To reconstruct an image of δμₐ(r) from Eq. 2a, L₀ and G are computed for a homogeneous background of known optical properties using a Monte Carlo simulation in a focused beam geometry (Dunn et al., Applied Optics, 35:3441 (1996). The Green function is computed by simulating the propagation of light from the detector to the medium and then utilizing reciprocity to determine the fraction of light reaching the detector from direction Ω at point r in the medium. Once the background radiance and Green function have been computed for all source-detector pairs, an image of δμₐ(r) is reconstructed using the measured values, L₁, which are obtained here for simulated data.

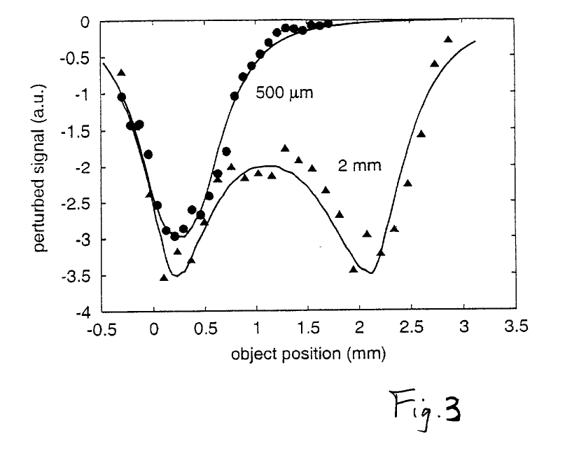

Before using Eq. 2a to reconstruct an image, the validity of the first Born approximation for the transport equation must be established. To test the validity, a perturbed signal computed using Eq. 2a was compared with a perturbed signal computed using a perturbative Monte Carlo simulation of an absorbing object embedded in a homogeneous background (Sassarolia et al., Applied Optics, 37:7392, 1998). The absorbing object used in the simulated comparison was a cubic object with dimensions of 100 μm located at a depth of 1 mm beneath the surface of a turbid sample. The optical properties programmed for the simulation of the background and perturbation were μₐ=0.001 mm⁻¹, μₛ=10 mm⁻¹, g=0.9, and δμₐ=0.1 mm⁻¹. The perturbed signal computed with Eq. 2a and with the full perturbative Monte Carlo model as the absorbing object was laterally translated through the sample are plotted in FIG. 3, which shows the comparison of the perturbed signal computed with the first Born approximation (solid lines) and with a full perturbative Monte Carlo simulation (symbols) at source-detector separations of 500 μm and 2 mm for an object located 1 mm beneath the surface with δμₐ=0.1 mm⁻¹. A decrease in the perturbed signal is observed while either the source or detector is scanned across the object position. Although a useful image can be reconstructed using one source-detector pair at a fixed separation distance (offset), image resolution improves as additional source-detector pairs, at different offsets, are added to the calculation.

The amplitudes of the perturbed signals in FIG. 3 have not been normalized and demonstrate that the absolute amplitudes of the perturbed signals are accurately predicted using the linear perturbation model.

The comparison illustrates that the first Born approximation accurately predicts the perturbed signal and that Eq. 2a can be used to reconstruct an image of δμₐ(r). Since the first Born approximation is a linear approximation, the magnitude of the perturbed signal is directly proportional to the magnitude of δμₐ. Based on the simulations, the linear approximation begins to deviate from the value predicted by the full perturbative Monte Carlo model at a δμₐ of about 1.5 mm⁻¹ for a 100 μm object located at a depth of 1 mm. In general, the value at which the linear approximation breaks down will vary depending on the size and depth of the perturbation. Results indicate that the maximum δμₐ increases as the depth of the object increases and decreases as the physical size of the perturbation increases, in agreement with standard perturbation theory.

### Indirect Mode Imaging Devices

The new indirect mode imaging systems include (i) an illumination and detection device, such as a standard scanning confocal microscope (e.g., a Zeiss, LSM 410®), or an endoscopic probe, with various components that are used to illuminate a sample and collect light emitted from the sample, such as from tissue in a patient or animal body; (ii) a light source that is connected to the illumination and detection device by optic fibers; and (iii) an apparatus that converts the light into a digital signal and includes a processor programmed with algorithms that can process the digital signal into indirect, three-dimensional images that provide information about features within a sample, such as diagnostic and prognostic information about a tissue sample in a living animal or human patient.

The new methods can be carried out with devices that are similar in some respects to confocal microscopes, but with a variable offset between the light source and one or more detectors. For example, the system can include an apparatus that is connected to a standard scanning confocal microscope. The light source can be a laser, such as a diode laser. Other light sources include light emitting diodes and lamps. Both visible and near-infrared light can be used, e.g., in the wavelength range of 400 to 1500 nm, but the choice of wavelength depends to some extent on the nature of the sample and the features to be imaged within the sample. For example, to image hemoglobin within blood vessels, the preferred wavelength range is about 570 to 650 nm. To image ceramics, wavelengths of from 1300 to 1500 nm can be used to minimize scattering from the sample. The light can be from a continuous wave (CW) source with a set amplitude and frequency, or can be modulated in either or both amplitude and/or frequency. In addition, the light can be pulsed light. Incoherent light sources such as mode-locked solid-state pulsed lasers, and super-luminescent diodes can provide higher spatial resolution by providing optical measurements that are path-length resolved.

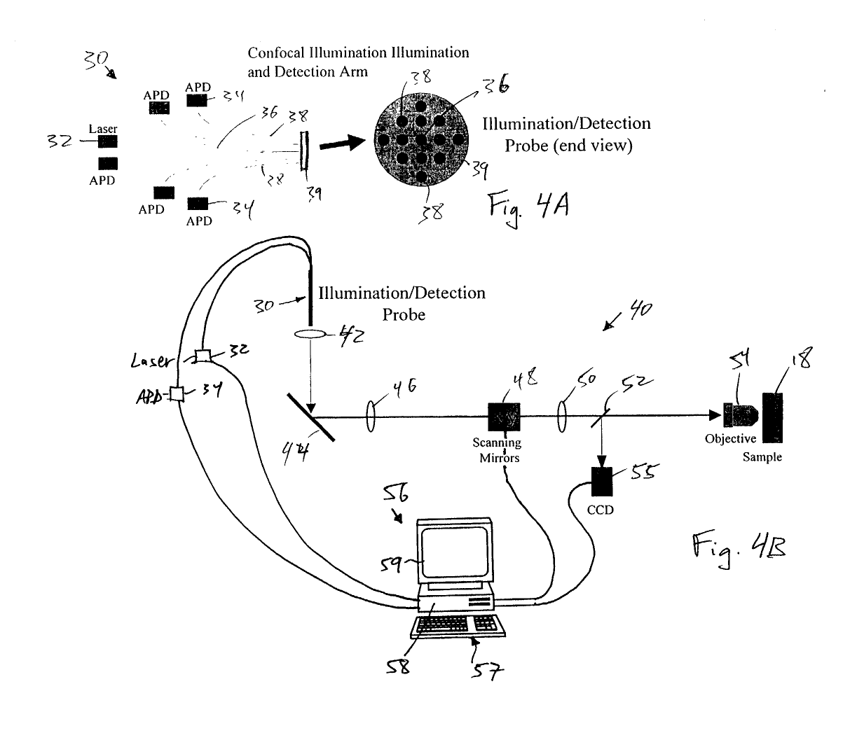

FIG. 4A is a schematic end view of a confocal illumination/detection arm or probe 30 useful in the new imaging systems. Probe 30 utilizes a modified form of a traditional confocal microscope. Probe 30 is connected to a light source 32 such as a laser, and multiple photodetectors 34, such as avalanche photodiodes (APDs) or photomultiplier tubes (PMTs) with different offsets from the optical axis. This configuration can be achieved through an array of optical fibers 36, 38 collected in a distal endplate 39. Source fiber 36, which can be located in the center of the probe or can be arranged to one side, delivers the light to the sample and also serves as a traditional confocal pinhole. The remaining detector fibers 38 serve as off-axis pinholes and each is coupled to a photodetector 34. The fiber diameter and amount of offset can be varied to select signal level and penetration depth within the medium. For example, the average penetration depth of the detected light is about 0.5 mm for a source-detector separation of 750 mm and a fiber diameter of 100 mm. In addition, the offsets for some detector fibers to the source fiber can differ from the offsets for other detector fibers to the source fiber. In fact, the best images are obtained by having a multiplicity of source-detector separations.

The new instruments 30 can be used in an imaging system 40 shown in FIG. 4B. As shown in FIG. 4B, illumination/detection probe 30 directs light to mirror 44 through lens 42, and then from the mirror through a second lens 46. The light continues through x/y scanning mirrors 48 and lens 50 and is scanned in a user-specified pattern (typically a raster scan) in the back focal plane of the objective lens 54. This scanning process is controlled by processor 56 using standard scanning confocal microscope scanning protocols. A portion of the light passes through a beam-splitter 52 and to objective 54, which focuses the light on to sample 18. Light reflected from the sample, returns along the same path, but some of the reflected light is directed to an imaging device 55, such as a charge-coupled device (CCD) or camera, that provides an image of the surface of the sample. The direct image of the surface of the sample can be co-registered or superimposed over the image provided by the new IMI. The remainder of the reflected light passes back to the illumination/detection probe 30, to be processed to reconstruct an image of features within sample 18 by processor 56.

Probe 30 of FIG. 4A is used with an apparatus that includes a standard analog-to-digital converter and a processor, as described in further detail below. Both the A-to-D converter and the processor can also be in a separate PC. As shown in FIG. 4B, such a processor 56 generally includes an input/control device 57, a memory 58, and an output device 59. The processor can be an electronic circuit comprising one or more components. The processor can be implemented in digital circuitry, analog circuitry, or both, it can be implemented in software, or may be an integrated state machine, or a hybrid thereof. Input/control device 57 can be a keyboard or other conventional device, and the output device 59 can be a cathode ray tube (CRT), other video display, printer, or other image display system. Memory 58 can be electronic (e.g., solid state), magnetic, or optical. The memory can be stored on an optical disk (e.g., a CD), an electromagnetic hard or floppy disk, or a combination thereof.

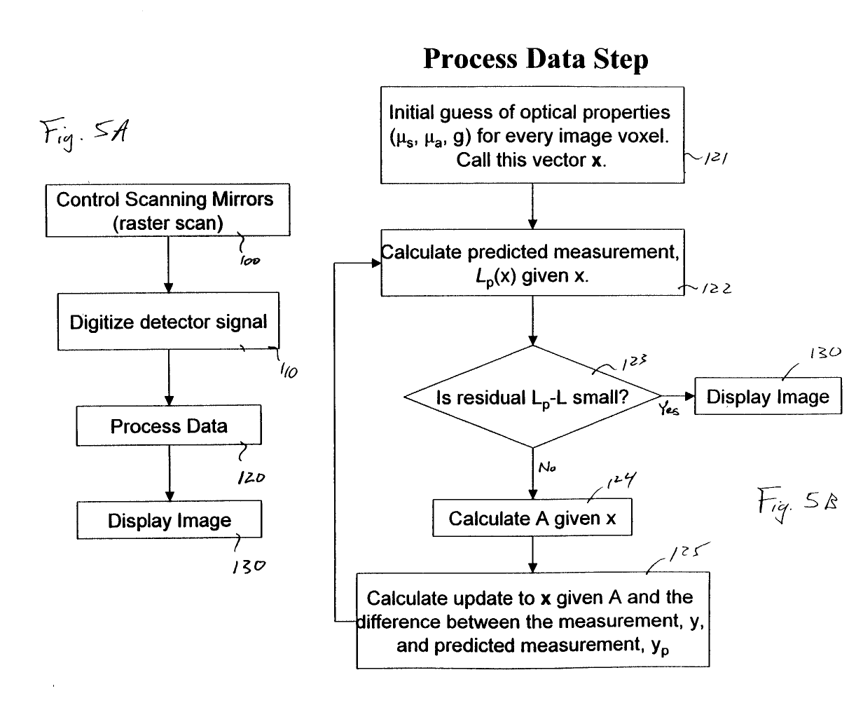

As shown in the flowchart of FIG. 5A, the processor controls data acquisition and processing. First, in step 100, the processor controls the scanning of the mirror 44 to illuminate the sample, then, optionally, amplifies the detector signals to an appropriate level with analog circuitry. In step 110, the analog signal is digitized with an analog-to-digital converter, which may be contained within a computer. The digitized detector signal is stored in computer memory or on a hard disk. In step 120, the digitized detector signals are processed using an image reconstruction software package, which is described in further detail below. The resulting three-dimensional image is then displayed, e.g., in two-dimensional form (step 130).

To reconstruct an image from the digitized detector signals, Eqs. 2a and/or 2b must be solved for the terms δμₐ and/or δμₛ at each voxel location. The flowchart in FIG. 5B illustrates the steps in data processing step 120. The digitized data is processed by expressing Eq. 2a as a matrix equation of the form y=Ax, where x=δμₐ, is the vector of optical properties at each voxel, y is the vector of detector signals for each source-detector pair, and A₁₃ is an element of matrix A representing the product of the Green function for the transport equation (G) and the first-order (background) detector signal (L₀) for each measurement i at each voxel j. The Green function is obtained through Monte Carlo simulations of the light propagation in the medium for a particular source-detector configuration (Dunn et al., Applied Optics, 35:3441 (1996)). This matrix equation can then be solved using standard techniques (see Arridge Inverse Problems, 15(2):R41, 1999), such as truncated singular value decomposition.

In step 121, the processor makes an initial “guess” of the optical properties (μₛ, μₐ, g) for every image voxel. This list of optical properties at each voxel is defined as vector x. Next, in step 122 the processor calculates a predicted detector signal measurement, Lₚ(x) given x. In step 123, the processor queries whether residual Lₚ−L is above or below a threshold level typically set at the measurement noise level of the system. The measurement noise should typically be less than about 0.1% of the measurement signal (Lₚ−L/L<10⁻³). If the residual is less than the threshold, then the image is displayed (step 130). If the residual is larger than the threshold, then the matrix A is calculated given x (step 124). In step 125, the processor calculates an update to x given A (using any of several known techniques) and the difference between the measurement y and predicted measurement yp and uses the updated x to calculate another predicted detector signal measurement, Lₚ(x) given the new x (step 122).

The process steps 122, 123, 124, and 125 are repeated until the residual Lₚ−L is small enough, in which case an image is displayed (step 130).

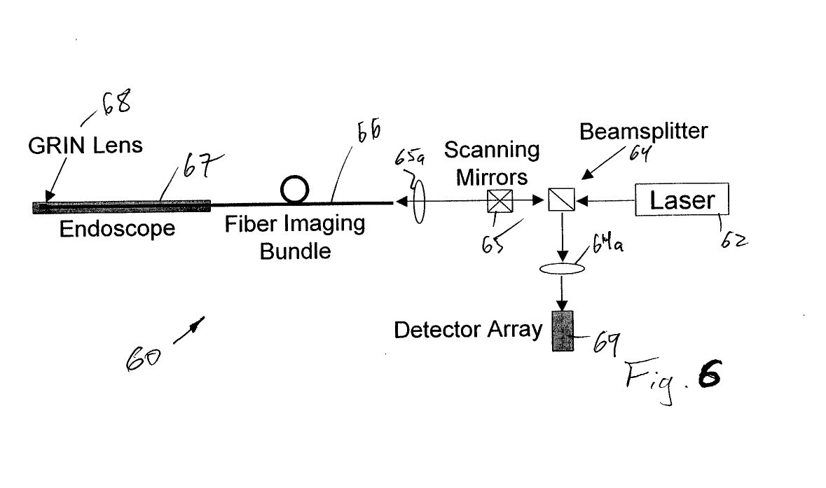

FIG. 6 shows an endoscopic illumination/detection probe 60 for use in the new methods. In this probe 60, a laser 62 directs light through a beam-splitter 64, and through a pair of scanning mirrors 65. Light is focused through lens 65a onto the face of a fiber optic imaging bundle 66. A focused spot of light is scanned across the fiber bundle to couple light into a single fiber at a time. Fiber bundle 66 can be inserted into an endoscope 67 and a gradient index (GRIN) lens 68 can be used to focus the light exiting each fiber within the bundle to a point inside a sample, e.g., inside tissue. Other combinations of lenses, such as micro-lenses and holographic lenses, can be used to focus the light within the tissue.

Reflected light is also collected by GRIN lens 68 and is coupled back into the fiber bundle 66. The light exiting the bundle is imaged onto detector array 69 via lenses 65a and 64a, and through scanning mirrors 65 and beam-splitter 64a. The detector array 69 consists of one or more detector elements whose position is laterally displaced from the optical axis. The amount of displacement determines the penetration depth of the detected light. For example, a source-detector separation of 750 μm provides a penetration depth of about 500 μm. This displacement can be varied to change the penetration depth of the light as it passes into the tissue. By increasing the separation distance, the penetration depth increases (but the resolution decreases).

### Applications

The new methods and devices provide the ability to image through several millimeters of turbid samples, such as human and animal tissue, with a resolution of a few hundred microns, and can therefore be used to address many new biomedical problems and applications.

For example, the new methods and devices can be used to image subsurface blood vessels, e.g., through the skin or tissue, located several millimeters within tissues since the size of the vessels is a few hundred microns and the difference in absorption between the blood vessels and the surrounding tissue will be about 1 mm⁻¹ in the near infrared. In another example, the endoscopic probe can be used to image walls of larger blood vessels, as well as the tissue outside the blood vessel walls.

Another use of the new methods is in optimizing the use of the indirect mode of the scanning laser ophthalmoscope (Webb et al., Applied Optics, 26:1492, 1997) for imaging sub-retinal structures where an annular aperture is used in place of a confocal aperture so that multiply scattered light is detected. Currently the use of the indirect mode is based on observation rather than a theory or model based on light scattering in tissue. Therefore, the model presented in this paper could be applied to optimize its use, because it provides a method for quantifying spatially varying tissue optical properties (absorption and scattering). Quantitative knowledge of these optical properties may reveal useful diagnostic information about the tissue state.

### Software Implementation

The processing of the digital data corresponding to the light emitted from the sample can be implemented in hardware or software, or a combination of both. The method can be implemented in computer programs using standard programming techniques following the methods, equations, and figures described herein. Program code is applied to enter data to perform the functions described herein and to generate output information. The output information is applied to one or more output devices such as a display monitor.

Each program is preferably implemented in a high level procedural or object oriented programming language to communicate with a computer system. However, the programs can be implemented in assembly or machine language, if desired. In any case, the language can be a compiled or interpreted language.

Each such computer program is preferably stored on a storage medium or device (e.g., ROM or magnetic diskette) readable by a general or special purpose programmable computer, for configuring and operating the computer when the storage media or device is read by the computer to perform the procedures described herein. The computer program can also reside in cache or main memory during program execution. The processing methods can also be implemented as a computer-readable storage medium, configured with a computer program, where the storage medium so configured causes a computer to operate in a specific and predefined manner to perform the functions described herein.

### EXAMPLES

The invention is further described in the following examples, which do not limit the scope of the invention described in the claims.

### Example 1

### Study of Angular Dependence of Radiance

To study the angular dependence of the radiance, the integrand of Eq. 12 above was computed using a Monte Carlo simulation. The geometry for the simulation consisted of a beam focused at a depth of 500 μm beneath the surface with a numerical aperture of 0.4. The distribution of photons within the sample was computed for a sample with the following optical properties: μₐ=0.01 mm⁻¹, μₛ=10 mm¹, g=0.9, and n=1.0. The source-detector separation was set at 750 μm.

The Green function G(r, \hat{Ω}|r_{d}, \hat{Ω}_{d}) was computed from the results of the simulation by translating the computed photon distribution radially and utilizing the reciprocity of the photon paths, G(r, \hat{Ω}|r′, \hat{Ω}′)=G(r′, −\hat{Ω}|r, \hat{Ω}).

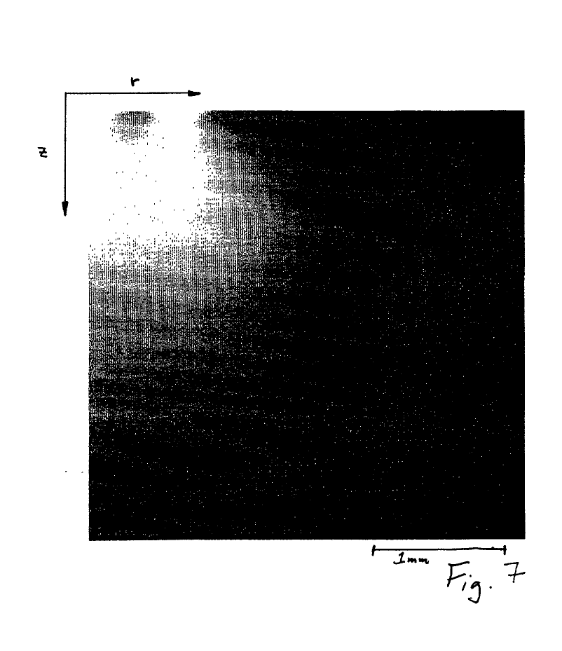

As an example, FIG. 7 shows the photon distribution within the medium for photons reaching the detector,

∫G(rₛ, Ωₛ|r, Ω)G(r, Ω|r_{d},Ω_{d})dΩ (3)

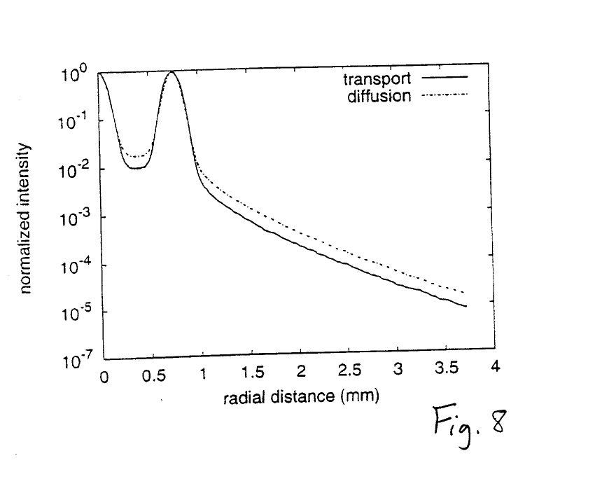

By examining the radial distribution of photons within the medium, the angular dependence can be illustrated as in FIG. 8, in which the radial distribution is plotted at a depth of 500 μm for the angular resolved case (Eq. 3; solid line labeled “transport”), and the case when the angular dependence is ignored (Eq. 4; dash-dotted line labeled “diffusion”),

∫G(rₛ, Ωₛ|r, Ω)dΩ∫G(r, Ω|r_{d}, Ω_{d})dΩ (4)

The intensity in FIG. 8 has been normalized to the peak value in each case for comparison, but the absolute intensity is approximately an order of magnitude smaller for the case where the angular dependence is accounted for (Eq. 3) than where the angular dependence is ignored (Eq. 4). As shown in FIG. 8, the dip in intensity between the source and detector locations is more pronounced when the angular dependence is considered. Therefore, when reconstructing an image of an absorption inhomogeneity, the angular dependence of the photon distribution should be taken into account.

### Example 2

### Simulation of Image Reconstruction

Once the validity of the first Born approximation has been established (FIG. 3), an image of δμₐ(r) can be constructed by writing Eq. 2 as a matrix equation of the form y=Ax, where y is the set of measurements and x=δμₐ(r). Truncated singular value decomposition was used to invert this set of equations and find δμₐ(r) using simulated data (Arridge, Inverse Problems, 15(2):R41, 1999).

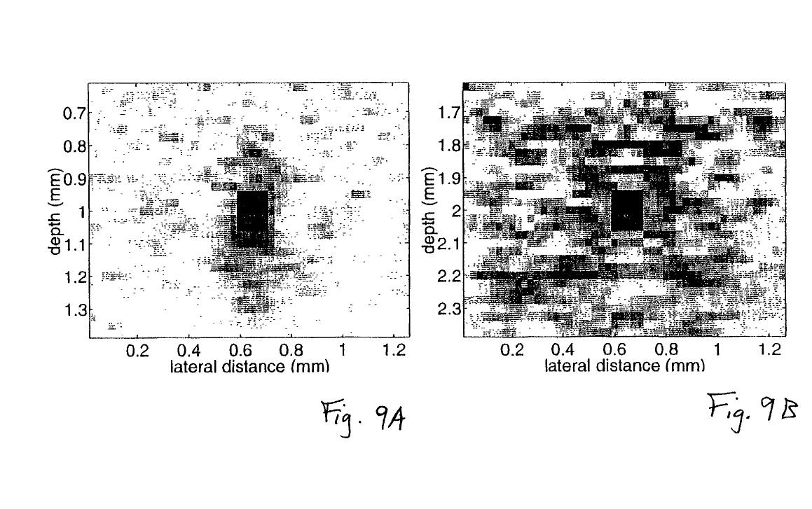

Reconstructed images of a 100 μm absorbing object (δμₐ1 mm⁻¹) at depths of 1 and 2 mm are shown in FIGS. 9A and B. The background optical properties of the medium were the same as those described in conjunction with FIG. 3 above. The set of measurements used in the simulated reconstruction consisted of source-detector separations ranging from 400 μm to 2 mm in 200 μm increments at numerical apertures of 0.2 and 0.4, for a total of 18 source-detector pairs (9 with a source and detector NA of 0.2 and 9 with an NA of 0.4). Each pair was translated 1.25 mm across the surface of the sample in 51 steps of 25 μm yielding a total of 918 measurements. The focal point of the source and detector was set to 1 mm beneath the surface for all measurements. The singular value spectrum was truncated at 250 and this number was determined by considering a potential signal to noise ratio of 103 for the measurements (although in the present simulation there is no noise). The system was assumed to be shot noise limited and the number of singular values was chosen such that the magnitude of the perturbed signal was greater than the measurement noise in the total detected signal (background+perturbation). The images clearly indicate that 100 μm axial and lateral resolution is maintained to a depth of 1 mm (FIG. 9A) and 2 mm (FIG. 9B).

Based on the images of FIGS. 9A and 9B, it is clear that this method is capable of imaging in the spatial regime lying between the microscopic and diffuse regimes.

### Other Embodiments

It is to be understood that while the invention has been described in conjunction with the detailed description thereof, the foregoing description is intended to illustrate and not limit the scope of the invention, which is defined by the scope of the appended claims. For example, the principles described herein can be applied for imaging other types of radiation that has been multiply scattered and detected, i.e., imaging at other wavelengths such as x-rays and microwaves given proper illumination/detection devices. Other aspects, advantages, and modifications are within the scope of the following claims.

What is claimed is:

1. A method of indirect mode imaging a sample, the method comprising
(a) illuminating the sample with optical radiation from a source;
(b) receiving optical radiation emitted from the sample with one or more detectors;
(c) digitizing the optical radiation received from the detectors to generate a digitized signal and transmitting the digitized signal to a processor; and
(d) processing the digitized signal to reconstruct an image of spatially varying optical properties of one or more features within the sample by performing a non-linear minimization between the digitized data and a transport-based photon migration model.

2. The method of claim 1, wherein the detectors are each located at a position offset from the source.

3. The method of claim 2, wherein one or more of the offsets of each detector-source pair are different.

4. The method of claim 1, wherein the image δμₐ(r) is reconstructed by calculating a matrix equation of the form y=Ax, wherein
μₐ is the absorption coefficient,
r is a position within the sample,
x=δμₐ,
y is the vector of detector signals for each source-detector pair, and
Aᵢⱼ is an element of matrix A representing the product of (i) a Green function:
G(r, \hat{Ω}|r_{d}, \hat{Ω}_{d}), for a transport equation (G): μₐ(r)=μ⁰ₐ+δμₐ(r) and μₛ(r)=μ⁰ₛ+δμₛ(r), and (ii) a first-order, background detector signal (L₀), for each measurement i at each voxel j.

5. The method of claim 1, wherein the optical radiation is near-infrared radiation.

6. The method of claim 1, wherein the optical radiation is scanned from the source across the sample to simulate multiple sources.

7. The method of claim 1, wherein ten detectors are used to receive optical radiation from the sample.

8. The method of claim 2, wherein the offset is from 0.1 to 10 mm.

9. The method of claim 3, wherein the one or more offsets are from 0.1 to 10 mm.

10. The method of claim 1, wherein the sample contains an absorbing object, and the image δμₐ(r) is reconstructed by solving Equation 2a:
L₁(r_{d}, \hat{Ω}_{d})=−∫∫δμₐ(r)L₀0(rₛ, r, \hat{Ω})G(r, \hat{Ω}|r_{d}, \hat{Ω}_{d})d\hat{Ω}d³r.

11. The method of claim 1, wherein the sample contains a scattering object, and the image δμₛ (r) is reconstructed by solving Equation 2b:
L₁(r_{d}, \hat{Ω}_{d})=−∫∫δμₛ(r)[L₀(rₛ, r, \hat{Ω}) ∫L₀(rₛ, r, \hat{Ω}′)p(Ω, Ω′)dΩ′]G(r, \hat{Ω}|r_{d}, \hat{Ω}_{d})d\hat{Ω}d³r.

12. The method of claim 1, wherein the sample contains an absorbing and scattering object, and the image δμₐ(r)+δμₛ(r) is reconstructed by solving the summation of Equations 2a+2b.

13. A system for indirect imaging of a sample comprising
(a) a probe comprising a source optic fiber, one or more detector optic fibers, and a distal end, wherein a distal end of the source optic fiber and a distal end of each of the detector optic fibers extends through and ends in the distal end of the probe;
(b) an optical radiation source connected with a proximal end of the source optic fiber;
(c) one or more photodetectors, each connected to a proximal end of one of the detector optic fibers, to receive and convert optical radiation from each detector optic fiber into a digital signal corresponding to light emitted from the sample; and
(d) a processor that processes the digital signal produced by the photodetectors to provide on an output device an image of spatially varying optical properties of one or more features within the sample, wherein the image is reconstructed by performing a non-linear minimization between the digitized data and a transport-based photon migration model.

14. The system of claim 13, wherein the distal end of each detector optic fiber is offset from the distal end of the source optic fiber.

15. The system of claim 14, wherein the offset is from 0.1 to 10 mm.

16. The system of claim 13, wherein the processor is programmed to process the digital signal to provide an image δμₐ(r) that is reconstructed by calculating a matrix equation of the form y=Ax, wherein
μₐ is the absorption coefficient,
r is a position within the sample,
x=δμₐ,
y is the vector of detector signals for each source-detector pair, and
Aᵢⱼ is an element of matrix A representing the product of (i) a Green function:
G(r, \hat{Ω}|r_{d}, \hat{Ω}_{d}), for a transport equation (G): μₐ(r)=μ⁰ₐ+δμₐ(r) and μₛ(r)=μ⁰ₛ+δμₛ(r), and (ii) the first-order (background) detector signal (L₀), for each measurement i at each voxel j.

17. The system of claim 14, wherein one or more of the offsets of each detector-source pair are different.

18. The system of claim 13, wherein the optical radiation is near-infrared radiation.

19. The system of claim 13, wherein the optical radiation is scanned from the source optic fiber across the sample to simulate multiple sources.

20. The system of claim 13, wherein ten detectors are used to receive optical radiation from the sample.

21. The system of claim 14, wherein the offset is from 0.1 to 10 mm.

22. The system of claim 17, wherein the one or more offsets are from 0.1 to 10 mm.

23. The method of claim 1, wherein the image is three-dimensional.

24. The system of claim 13, wherein the image is three-dimensional.

25. A probe for indirect mode imaging, comprising a source optic fiber, one or more detector optic fibers, and a distal end, wherein a distal end of the source optic fiber and each of the detector optic fibers extends through and ends in the distal end of the probe, and wherein the distal end of the source optic fiber is offset from each distal end of the detector optic fibers by 0.1 to 10 mm.

26. The probe of claim 25, further comprising a scanning mirror to scan optical radiation across a sample.

27. The probe of claim 25, wherein one or more of the offsets of one or more source-detector pairs are different.
